(19) United States
(12) Patent Application Publication
(10) Pub. No.: US 2025/0185626 A1
(43) Pub. Date: Jun. 12, 2025

(54) SCIENTIFIC DEVICE FOR THE STUDY OF ANIMALS, IN PARTICULAR MARINE ANIMALS

(71) Applicants: Blancpain SA, Le Brassus (CH); Andromède Océanologie Sàrl, Mauguio (FR)

(72) Inventors: Yves WINKLER, Schmitten (CH); Timothée HALLER, Rochefort (CH); Yannick VONLANTHEN, La Chaux-de-Fonds (CH); Antonin GUILBERT, Montpellier (FR); Laurent BALLESTA, Mauguio (FR)

(73) Assignees: Blancpain SA, Le Brassus (CH); Andromède Océanologie Sàrl, Mauguio (FR)

(21) Appl. No.: 18/915,546

(22) Filed: Oct. 15, 2024

(30) Foreign Application Priority Data
Dec. 7, 2023 (EP) 23215078.9

(51) Int. Cl. A01K 11/00; A01K 29/00

(52) U.S. Cl. CPC A01K 11/006; A01K 29/005

(57) ABSTRACT

A device for the study of animals, which device can be launched at an animal for tagging and sample collection from the animal, which device includes an arrow including the following elements, which are coaxial and removable: an arrow body, a hollow cutting dart and an insert, the hollow cutting dart including, in a rear part, support means for a propulsion device and carrying, in a front part, this hollow cutting dart capable of penetrating the skin of an animal on impact and of coring a sample of the animal in a bore, this hollow cutting dart externally carrying, bearing against a first front shoulder of the arrow body, the insert to be fixed under the skin of the animal, the arrow body including, at the rear part, a second shoulder for stopping the arrow body from penetrating into the animal.

### TECHNICAL FIELD OF THE INVENTION

The invention relates to a scientific device for the study of animals, which device is arranged to be launched by a propulsion device at an animal for tagging and sample collection from said animal, which device comprises an arrow.

The invention relates to the field of scientific experimentation on live animals, and more particularly on marine animals.

### TECHNOLOGICAL BACKGROUND

Placing a tracking tag on a live animal is a delicate and difficult operation. Scientists are looking to avoid having to put an animal to sleep, or having to catch a marine or aquatic animal, in order to fit a tag. This is to avoid subjecting the animal to significant stress.

The same applies to tissue sampling, which is particularly difficult on wild animals, especially highly sensitive species such as sharks. The purpose of taking tissue samples is to determine the animal's state of health, as well as any pathologies or genetic anomalies.

Trials have been successfully conducted on carrying out tagging or sampling operations directly in the environment where the animal lives, for example in the marine environment, and at a reasonable distance from the animal, using a special propulsion device, to significantly reduce the stress caused by the operation. Other trials have been conducted to combine the tag-fitting and sample-taking operations so as to limit the investigation to a single contact with the animal, again with the aim of reducing the impact on its well-being. In particular, prior art discloses an arrow that allows a tag to be fitted and a biological sample to be taken from such species at the same time. However, the method is not infallible, as skin penetration often does not occur, which means that neither tagging nor sample collection is possible, or the animal's flesh does not adhere well to the sampling tube, although tagging occurs correctly.

The aim is therefore to improve the reliability of an experimental device so that a tag can be fitted and a reliable sample collected in a single operation and without fail.

### SUMMARY OF THE INVENTION

The aim of the invention is to improve the success rate of combined tagging/sampling shots at animals, more particularly marine animals, even more particularly sharks, such as hammerhead sharks.

To this end, the invention relates to a scientific device for the study of animals, which device is arranged to be launched by a propulsion device at an animal for tagging and sample collection from said animal, which device comprises an arrow.

According to the invention, said arrow comprises the following elements, which are substantially coaxial about a longitudinal direction and removable from one another: an arrow body, a hollow cutting dart and at least one insert, said arrow body comprising, in a rear part, support means for a said propulsion device and longitudinally supporting, in a front part, said hollow cutting dart forming a sampling tube intended to penetrate the skin of an animal on impact and to core a sample of said animal in a bore in said hollow cutting dart, said hollow cutting dart externally carrying said at least one insert intended to be fixed under the skin of said animal, said at least one insert being removable from said arrow body and from said hollow cutting dart and intended to bear against a first front shoulder of said arrow body, which arrow body comprises, at said rear part, a second shoulder intended to stop said arrow body from penetrating into said animal.

### BRIEF DESCRIPTION OF THE FIGURES

The purposes, advantages and features of the invention will be better understood upon reading the following detailed description given with reference to the accompanying drawings, in which:

FIG. 1 diagrammatically shows a perspective view of a scientific device for studying animals, which device comprises, shown from left to right in the figure, propulsion means arranged to project an arrow at an animal observation and/or tagging zone, withdrawal means for recovering this arrow after a sample has been taken, and an arrow specific to the invention, in a plurality of assembled parts, and comprising, from back to front, an arrow body and a hollow cutting dart, forming a receptacle for a sample, and which carries an insert intended to be inserted under the animal's skin, this insert carrying a penetration cone comprising resilient blades shown here deployed in a free configuration of the arrow, which are flattened along the hollow cutting dart when the arrow enters the animal's body in a stressed position, and which then work to spread radially, preventing the insert from coming out once inserted;

FIG. 2 diagrammatically shows an elevation view of the arrow in FIG. 1;

FIG. 3 diagrammatically shows a sectional view, taken axially along the cutting plane AA in FIG. 2, of a first alternative embodiment of the arrow in FIG. 1, wherein the hollow cutting dart is fastened to the arrow body by a screwed connection;

FIG. 4 diagrammatically shows a close-up sectional view, taken axially, of a second alternative embodiment of the arrow in FIG. 1, wherein the hollow cutting dart is fastened to the arrow body by at least one radial assembly element.

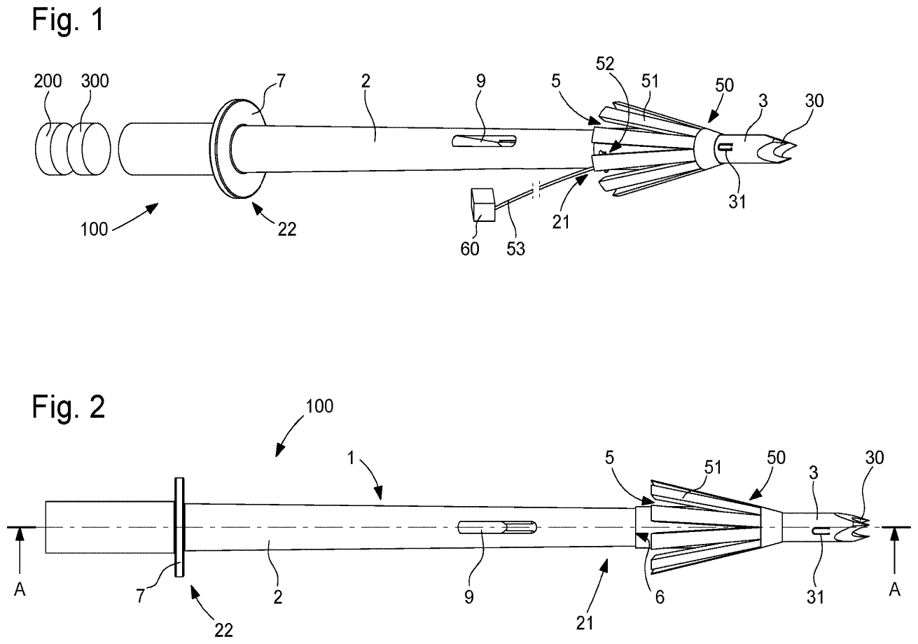

### DETAILED DESCRIPTION OF THE INVENTION

In order to allow tagging and sample collection without causing major injury to the animal, the invention relates to a scientific device 100 for the study of animals, which device is arranged to be launched by a propulsion device at an animal for tagging and sample collection from that animal, which device comprises an arrow 1.

This arrow 1 comprises a rigid part forming a support, referred to as the arrow body 2, and a removable tip. The support is the structural part used for projecting the arrow 1 and for withdrawing it after a sample has been collected. The removable tip, referred to as the hollow cutting dart 3, fulfils two functions: penetrating the animal's body and collecting a sample.

Preferably, as shown in the figures, the removable tip carries the removable insert; in another alternative embodiment, not illustrated, the arrow body carries the removable insert.

The term “insert” is understood to mean, in the broadest sense, any insert intended to remain in the animal's body, for example as illustrated in the figures, an attachment element 52 for attaching a cable 53 towing a tag 60, in particular a radio position transmitting tag, or directly such a tag permanently inserted in the animal's body, and/or a sensor, but also for example an element allowing the animal's wound to be treated and/or closed; thus two or more inserts, arranged one behind the other on the removable tip, can be inserted into the animal in a single operation.

According to the invention, this arrow 1 thus comprises, substantially coaxial around a longitudinal direction D, and removable from one another, an arrow body 2, a hollow cutting dart 3 and at least one insert 5.

The arrow body 2 comprises, in a rear part 22, support means for a propulsion device. In a front part 21, it supports the hollow cutting dart 3 longitudinally.

This hollow cutting dart 3 forms a sampling tube intended to penetrate the skin of an animal on impact and core a sample of the animal's tissue in a bore 4 of the hollow cutting dart 3.

More particularly, the hollow cutting dart 3 externally carries at least one insert 5, which is arranged to be fixed under the animal's skin. At least one of these inserts 5, or the insert 5 if only one is present as illustrated in the figures, can be removed from the arrow body 2 and from the hollow cutting dart 3, and is arranged to rest against a first front shoulder 6 of the arrow body 2.

The arrow body 2 has a second shoulder 7 at the rear part 22, which shoulder is intended to stop the arrow body 2 from penetrating the animal.

More particularly, the insert 5 carries a penetration cone 50 outside the hollow cutting dart 3, comprising resilient blades 51 which are arranged to deploy radially under the animal's skin and prevent the insert 5 from coming away after being inserted beneath the animal's skin. These resilient blades, in the free state, form a fanned-out cone, as seen in FIG. 1. These resilient blades 51, which are deployed when the arrow is in a free configuration, are flattened along the hollow cutting dart when the arrow enters the animal's body in a stressed position, and then work to spread radially, preventing the insert from coming out once inserted.

More particularly, the penetration cone 50 is made of plastics material. Advantageously, this penetration cone 50 is stopped longitudinally by the attachment element 52, or a radial lug, or the like, comprised in the insert 5.

In a particular alternative embodiment, the insert 5 is made of biodegradable material, or biodegradable plastics material, to release the tag 60 after a set period of time.

In another alternative embodiment, the body of the tag 60 is made of biodegradable material, or biodegradable plastics material, to release the tag 60 after a set period of time.

In another alternative embodiment, the cable 53 towing the tag 60 is made of biodegradable material, or biodegradable plastics material, to release the tag 60 after a set time.

More specifically, each insert 5 is mounted with a close sliding fit on the hollow cutting dart 3.

More particularly, the arrow body 2 comprises front fastening means 23 for the reversible fastening of the hollow cutting dart 3, which comprises complementary front fastening means 32, and comprises, in the extension of the bore 4, a discharge channel 8 arranged for the discharge of liquid through at least one discharge opening 9, in the immediate vicinity of the front fastening means 23. Each discharge opening 9 extends obliquely from the discharge channel 8 towards the periphery of the arrow body 2, from the distal end of the hollow cutting dart 3 towards the rear part 22 of the arrow body 2, and is open in the discharge direction to facilitate this. The arrow 1 is thus well adapted to the case of marine animals, and has a very short water discharge route, which reduces the overpressure in the tube when the hollow cutting dart 3 penetrates the animal's skin, and thus increases the probability of tissue collection and the quantity collected. The alternative embodiment illustrated in the figures has three such discharge openings 9 for water discharge, which are open in the discharge direction to facilitate this.

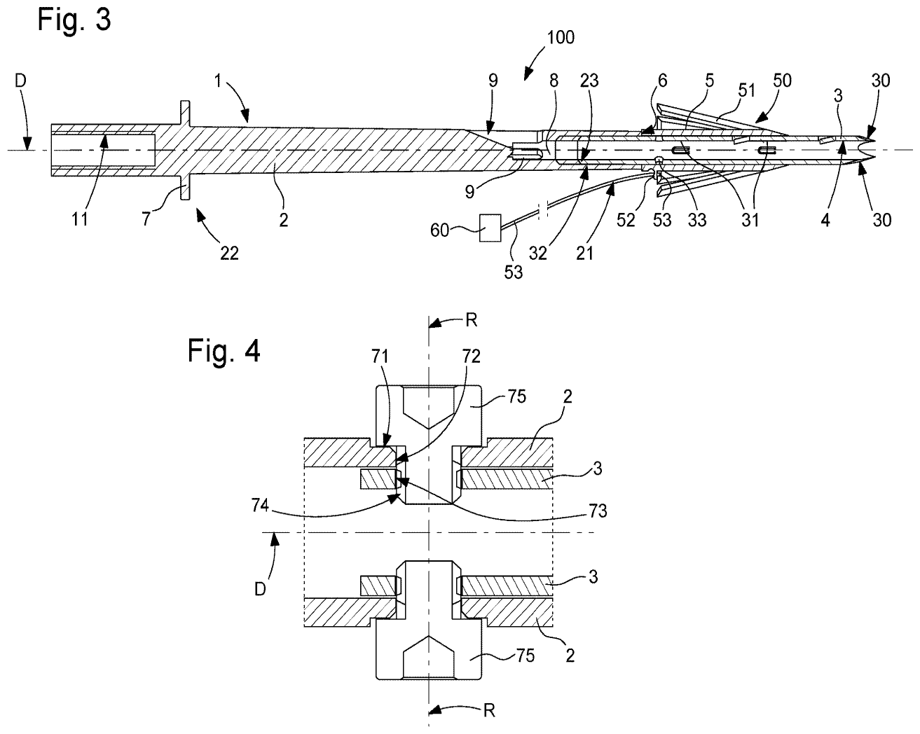

More particularly, in the first alternative embodiment in FIG. 3, the front fastening means 23 comprise an internal thread arranged to cooperate in a complementary manner with one of the complementary front fastening means 32, in particular an external thread on the hollow cutting dart 3, which comprises at least one manoeuvring notch 33, for example a radial hole, to make it easier to screw or unscrew.

More particularly, in the second alternative embodiment in FIG. 4, the hollow cutting dart 3 is fastened to the arrow body 2 by at least one radial assembly element, such as a radial screw 75, a pin or the like. For example, the front fastening means 23 of the arrow body 2 comprise at least one implant comprising a radial bore 72, in a radial direction R, and a supporting counterbore 71, and the complementary front fastening means 32 comprise at least one radial internal thread 73 in a radial direction R; the hollow cutting dart 3 and the arrow body 2 are then made integral with one another by at least one radial screw 75 comprising an external thread 74, arranged to bear against a supporting counterbore 71, and to cooperate with an internal thread 73, through a radial bore 72. FIG. 4 shows two such opposing radial screws 75. This second alternative embodiment avoids weakening the hollow cutting dart by axial threading.

More particularly, the arrow 1 has, in the rear part 22 and opposite the hollow cutting dart 3, rear fastening means 11 for fastening a pole or cable for retrieving the arrow 1.

More particularly, the hollow cutting dart 3 has a plurality of internal barbs 31, and/or the bore 4 of the hollow cutting dart 3 has a rough inner profile, for holding the animal sample when the arrow 1 is being withdrawn. Even more particularly, as can be seen in FIG. 3, the hollow cutting dart 3 has a plurality of internal, helically-arranged barbs 31, in order to minimise mechanical weakening. The presence of barbs 31 entering the tube at the tip makes it possible to retain the tissues when the arrow 1 is being retracted.

More particularly, the hollow cutting dart 3 has cutting edges 30 at its distal front part, arranged to perforate the animal's skin. Even more particularly, the hollow cutting dart 3 has at least three cutting edges 30 at its front part to ensure oblique penetration under the animal's skin. This design with three cutting edges at the end of the tip ensures that one cutting edge touches the skin first, whatever the orientation of the arrow 1 when it strikes the animal's skin obliquely.

More particularly, the hollow cutting dart 3 is interchangeable and is made of a material suitable for penetrating the skin of the type of animal concerned. It constitutes a removable interchangeable tip allowing the material of the tip to be chosen in order to maximise its mechanical properties and thus reduce the risks of deformation when penetrating the skin. The internal thread 23 in the arrow body 2 is preferably not flush at the end, so that the area of the hollow cutting body 3 that is subject to the greatest mechanical stress is not threaded, thus preventing it from weakening.

More particularly, the materials used to manufacture the arrow body 2 and the hollow cutting dart 3 are materials resistant to corrosion in a marine environment, such as stainless steel, titanium or the like. An example of an interesting material pairing is a 304 stainless steel arrow body 2 (corrosion resistance with good machinability) with a grade 5 titanium hollow cutting dart (very good corrosion resistance with high yield strength).

More particularly, the scientific device 100 for studying animals comprises propulsion means 200 intended to project the arrow at an observation and/or tagging zone of such an animal, and/or comprises withdrawal means 300 comprising a pole or a cable for recovering the arrow 1 enclosing the animal sample in the hollow cutting dart 3, after the insert 5 has been inserted under the animal's skin.

In short, the device described in the invention allows a tag to be fitted and a sample to be taken in a single operation and safely, while protecting the animal being studied as much as possible.

1. A scientific device for the study of animals, said device is arranged to be launched by a propulsion device at an animal for tagging and sample collection from said animal, said device comprises an arrow, wherein said arrow comprises the following elements, which are substantially coaxial about a longitudinal direction (D) and removable from one another: an arrow body, a hollow cutting dart and at least one insert, said arrow body comprising, in a rear part, support means for a said propulsion device and longitudinally supporting, in a front part, said hollow cutting dart forming a sampling tube configured to penetrate the skin of an animal on impact and to core a sample of said animal in a bore in said hollow cutting dart, said hollow cutting dart externally carrying said at least one insert configured to be fixed under the skin of said animal, said at least one insert being removable from said arrow body and from said hollow cutting dart and configured to bear against a first front shoulder of said arrow body, which arrow body comprises, at said rear part, a second shoulder configured to stop said arrow body from penetrating into said animal.

2. The scientific device for the study of animals according to claim 1, wherein said insert carries a penetration cone outside said hollow cutting dart, comprising resilient blades arranged to deploy radially under said animal's skin and prevent said insert from coming away after being inserted beneath said animal's skin.

3. The scientific device for the study of animals according to claim 2, wherein said penetration cone is made of plastics material.

4. The scientific device for the study of animals according to claim 1, wherein said insert comprises an attachment element for attaching a cable towing a tag.

5. The scientific device for the study of animals according to claim 4, wherein said insert and/or said cable and/or the body of said tag is made of biodegradable material, or biodegradable plastics material, to release said tag after a set period of time.

6. The scientific device for the study of animals according to claim 1, wherein each said insert is mounted with a close sliding fit on said hollow cutting dart.

7. The scientific device for the study of animals according to claim 1, wherein said arrow body comprises front fastening means for the reversible fastening of said hollow cutting dart, which comprises complementary front fastening means, and comprises, in the extension of said bore, a discharge channel arranged for the discharge of liquid through at least one discharge opening, in the immediate vicinity of said front fastening means, each said discharge opening extending obliquely from said discharge channel towards the periphery of said arrow body, from the distal end of said hollow cutting dart towards said rear part of said arrow body, and being open in the discharge direction to facilitate this.

8. The scientific device for the study of animals according to claim 7, wherein said front fastening means comprise an internal thread arranged to cooperate in a complementary manner with an external thread comprised in said hollow cutting dart, which comprises at least one manoeuvring notch to make it easier to screw or unscrew.

9. The scientific device for the study of animals according to claim 7, wherein said hollow cutting dart is fastened to said arrow body by at least one radial assembly element.

10. The scientific device for the study of animals according to claim 9, wherein said front fastening means of said arrow body comprise at least one implant comprising a radial bore, in a radial direction (R), and a supporting counterbore, and said complementary front fastening means comprise at least one radial internal thread in a radial direction (R); said hollow cutting dart and said arrow body thus being made integral with one another by at least one radial screw comprising an external thread, arranged to bear against a said supporting counterbore, and to cooperate with a said internal thread, through a said radial bore.

11. The scientific device for the study of animals according to claim 1, wherein said arrow has, in said rear part and opposite said hollow cutting dart, rear fastening means for fastening a pole or cable for retrieving said arrow.

12. The scientific device for the study of animals according to claim 1, wherein said hollow cutting dart has a plurality of internal barbs, and/or in that said bore of said hollow cutting dart has a rough inner profile, for holding said animal sample when said arrow is being withdrawn.

13. The scientific device for the study of animals according to claim 12, wherein said hollow cutting dart has a plurality of internal barbs arranged helically in said bore.

14. The scientific device for the study of animals according to claim 1, wherein said hollow cutting dart has cutting edges at its distal front part, arranged to perforate said animal's skin.

15. The scientific device for the study of animals according to claim 14, wherein said hollow cutting dart has at least three said cutting edges at its said front part to ensure oblique penetration under said animal's skin.

16. The scientific device for the study of animals according to claim 1, wherein said hollow cutting dart is interchangeable and is made of a material suitable for penetrating the skin of the type of animal concerned.

17. The scientific device for the study of animals according to claim 1, wherein said arrow body is made of 304 stainless steel and wherein said hollow cutting dart is made of grade 5 titanium.

18. The scientific device for the study of animals according to claim 1, wherein said scientific device for studying animals comprises propulsion means configured to project said arrow at an observation and/or tagging zone of such a said animal, and/or comprises withdrawal means comprising a pole or a cable for recovering said arrow enclosing said animal sample in said hollow cutting dart, after said insert has been inserted under said animal's skin.
